(19) United States
(12) Patent Application Publication
(10) Pub. No.: US 2009/0062400 A1
(43) Pub. Date: Mar. 5, 2009

(54) Method of treating glaucoma using rasagiline

(76) Inventors: Laurence Oron, Emek Hefer (IL); Cheryl Fitzer-Attas, Rehovot (IL); Ron Neumann, Ramat Hasharon (IL)

Correspondence Address: COOPER & DUNHAM, LLP, 30 Rockefeller Plaza, 20th Floor, NEW YORK, NY, 10112, US

(21) Appl. No.: 12/231,601

(22) Filed: Sep. 3, 2008

Related U.S. Application Data
(60) Provisional application No. 60/967,456, filed on Sep. 5, 2007.

(51) Int. Cl. A61K 31/136; A61P 27/06

(52) U.S. Cl. 514/657

(57) ABSTRACT

Disclosed is a method of reducing glaucoma in a subject afflicted with glaucoma, comprising administering to the subject an amount of R(+)-N-propargyl-1-aminoindan or a pharmaceutically acceptably salt thereof effective to reduce glaucoma.

This application claims the benefit of U.S. Provisional Application No. 60/967,456, filed Sep. 5, 2007, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Glaucoma is a group of ocular diseases characterized by progressive damage to the eye at least partly due to elevated intraocular pressure (IOP)(“Glaucoma”, Merck Manual of Diagnosis and Therapy (1999), Merck Research Laboratories, (Whitehouse Station, N.J.), 733-738). Additionally, glaucoma is characterized by retinal ganglion cell (RGC) death, axon loss and an excavated appearance of the optic nerve head (Alward, “Medical Management of Glaucoma”, N Eng J Med, 1998; 339:1298-1307). Glaucoma can be diagnosed before vision loss occurs by visual field testing and by opthalmoscopic examination of the optic nerve to detect “cupping.” The management of glaucoma is based on lowering the IOP to prevent further optic nerve damage. The mean IOP in normal adults is 15 to 16 mm Hg; the normal range is 10 to 21 mm Hg. The first step in the management of glaucoma is based on lowering the IOP using topically applied medications (Coleman, “Glaucoma”, Lancet, 1999; 354:1803-1810). Currently there are five major classes of medications that are used to lower the IOP: β-adrenergic antagonists, adrenergic agonists, parasympathomimetics, prostaglandin-like analogues and carbonic anhydrase inhibitors (Medeiros, et al., “Medical Backgrounders: Glaucoma”, Drugs of Today 2002; 38:563-570). Although most medications are applied topically to the eye, they can cause severe systemic side effects and adversely affect the quality of the patient's life. If additional lowering of IOP is indicated or if medication fails to sufficiently lower the IOP, laser trabeculoplasty is usually the next step. If IOP is still not adequately controlled, incisional glaucoma surgery is indicated (Id). The lowering of IOP, although significantly reducing the extent of neuronal loss, does not ensure cessation of the disease process, because the loss of Retinal Ganglion Cells (RGCs) may continue. Recent studies of the association between IOP regulation and visual field loss after medical or surgical intervention showed that ongoing neuronal loss reflected in visual field tests can be diminished if the IOP is low. However, neuronal loss may continue to occur after reduction of IOP (Bakalash, et al., “Resistance of Retinal Ganglion Cells to an Increase in Intraocular Pressure is Immunedependent”, Invest Opthalmol Vis Sci 2002; 43:2648-2653).

Glaucomatous optic neuropathy appears to result from specific pathophysiological changes and subsequent death of RGCs and their axons. The process of RGC death is thought to be biphasic: a primary injury responsible for initiation of damage followed by a slower, secondary degeneration attributable to the hostile environment surrounding the degenerating cells (Kipnis, et al., “T Cell Immunity To Copolymer 1 Confers Neuroprotection On The Damaged Optic Nerve: Possible Therapy For Optic Neuropathies”, Proc Natl Acad Sci 2000; 97:7446-7451).

RGC death mechanisms in experimental animal models of glaucoma and human glaucoma have been shown to involve apoptosis. Although the molecular mechanism triggering the apoptosis has not been identified, deprivation of neurotrophic factors, ischemia, chronic elevation of glutamate and disorganized nitric oxide metabolism are suspected to be possible mechanisms (Farkas, et al., “Apoptosis, Neuroprotection and Retinal Ganglion Cell Death: An Overview”, Int Opthalmol Clin 2001; 41:111-130). In addition, it is possible that the mechanisms leading to RGC death share common features with other types of neuronal injury, such as signaling by reactive oxygen species, depolarization of mitochondria, or induction of transcriptionally regulated cell death (Weinreb, et al., “Is Neuroprotection a Viable Therapy for Glaucoma?” Arch Opthalmol 1999; 117:1540-1544).

Rasagiline, R(+)—N-propargyl-1-aminoindan, is a potent second generation monoamine oxidase (MAO) B inhibitor (Finberg et al., Pharmacological properties of the anti-Parkinson drug rasagiline; modification of endogenous brain amines, reserpine reversal, serotonergic and dopaminergic behaviours, Neuropharmacology (2002) 43(7):1110-8). Rasagiline Mesylate in a 1 mg tablet is commercially available for the treatment of idiopathic Parkinson's disease as AZILECT® from Teva Pharmaceutical Industries, Ltd. (Petach Tikva, Israel) and H. Lundbeck A/S (Copenhagen, Denmark). See, also AZILECT®, Physician's Desk Reference (2006), 60^{th} Edition, Thomson Healthcare for the properties of rasagiline mesylate.

However, the effects of rasagiline on glaucoma patients cannot be predicted. For example, although the anti-inflammatory drugs acetylsalicylate and prednisolone are known to regulate the microglia responsible for the onset of photoreceptor apoptosis and retinal degeneration thereafter, both drugs proved to be unsuccessful. (Sarra et al., Effect of steroidal and non-steroidal drugs on the microglia activation pattern and the course of degeneration in the retinal degeneration slow mouse, Ophthalmic Res. (2005) 37(2):72-82)

Further, while deprenyl has been suggested for the treatment of glaucoma (Tatton, U.S. Pat. No. 5,981,598), deprenyl studies are not predictive of the effect of rasagiline. Rasagiline and deprenyl have been shown to exhibit different effectiveness in treating the same neurodegenerative disease. See, Lange et al., (1998) “Selegiline Is Ineffective in a Collaborative Double-blind, Placebo-Controlled Trial for Treatment of Amyotrophic Lateral Sclerosis” Arch. Neurol. 55:93-96 (Selegiline, i.e. 1-deprenyl, was ineffective in treating ALS); Compare with Waibel et al., (2004) “Rasagiline Alone and in Combination with Riluzole Prolongs Survival in an ALS Mouse Model” J. Neurol. 251(9):1080-4 (Rasagiline alone and in combination with riluzole was an effective treatment in the ALS mouse model).

The effects of rasagiline on glaucoma have not previously been studied.

### SUMMARY OF THE INVENTION

This subject invention provides a method of treating a subject afflicted with glaucoma, comprising administering to the subject an amount of R(+)-N-propargyl-1-aminoindan or a pharmaceutically acceptable salt thereof effective to treat the subject.

The subject invention also provides a pharmaceutical composition comprising R(+)-N-propargyl-1-aminoindan or a pharmaceutically acceptable salt thereof, an additional agent for treating glaucoma, and a pharmaceutically acceptable carrier.

The subject invention also provides a pharmaceutical composition for use in treating a subject afflicted with glaucoma, which comprises a therapeutically effective amount of R(+)-N-propargyl-1-aminoindan or a pharmaceutically acceptable salt thereof and a pharmaceutically acceptable carrier.

The subject invention also provides the use of R(+)-N-propargyl-1-aminoindan or a pharmaceutically acceptable salt thereof in the manufacture of a medicament for treating a subject afflicted with glaucoma.

The subject invention also provides a method for reducing retinal ganglion cell death in a subject in need thereof comprising administering to the subject an amount of R(+)-N-propargyl-1-aminoindan or a pharmaceutically acceptable salt thereof effective to treat the subject.

### DETAILED DESCRIPTION

This subject invention provides a method of treating a subject afflicted with glaucoma, comprising administering to the subject an amount of R(+)-N-propargyl-1-aminoindan or a pharmaceutically acceptable salt thereof effective to treat the subject.

In an embodiment, the amount of R(+)-N-propargyl-1-aminoindan or of the pharmaceutically acceptable salt thereof is from 0.01 mg to 20 mg per day.

In another embodiment, the amount of R(+)-N-propargyl-1-aminoindan or of the pharmaceutically acceptable salt thereof is from 0.5 mg to 5 mg per day.

In another embodiment, the amount of R(+)-N-propargyl-1-aminoindan or of the pharmaceutically acceptable salt thereof is 2 mg per day.

In another embodiment, the amount of R(+)-N-propargyl-1-aminoindan or of the pharmaceutically acceptable salt thereof is 1 mg per day.

In an embodiment of any of the preceding methods, the administration is of the pharmaceutically acceptable salt of R(+)-N-propargyl-1-aminoindan.

In an embodiment, the pharmaceutically acceptable salt is esylate, mesylate, sulfate or tartrate.

In a further embodiment, the pharmaceutically acceptable salt is mesylate.

In a further embodiment, the amount of R(+)-N-propargyl-1-aminoindan mesylate is 1.56 mg per day.

In an embodiment of any of the preceding methods, the administration is intraocular, ocular, oral, parenteral, periocular, rectal, systemic, topical or transdermal administration.

In a further embodiment, the administration is ocular.

In a further embodiment, the method of the administration is suitable for delivery into the posterior segment.

In a further embodiment, the method of administration is intraocular, periocular, systemic or topical.

In yet a further embodiment, the amount of R(+)-N-propargyl-1-aminoindan mesylate is from 0.01 mg to 2 mg per day.

In yet a further embodiment, the amount of R(+)-N-propargyl-1-aminoindan mesylate is from 0.1 mg to 1 mg per day.

In another embodiment, the R(+)-N-propargyl-1-aminoindan or the pharmaceutically acceptable salt thereof is in a pharmaceutical composition.

In another embodiment, the method further comprises administering to the subject an additional agent for treating glaucoma.

In another embodiment, wherein the additional agent for treating glaucoma is a β-adrenergic antagonist, adrenergic agonist, parasympathomimetic, prostaglandin-like analog, or carbonic anhydrase inhibitor.

The invention also provides a pharmaceutical composition comprising R(+)-N-propargyl-1-aminoindan or a pharmaceutically acceptable salt thereof, an additional agent for treating glaucoma, and a pharmaceutically acceptable carrier.

In an embodiment, the agent for treating glaucoma is a β-adrenergic antagonist, adrenergic agonist, parasympathomimetic, prostaglandin-like analog, or carbonic anhydrase inhibitor.

In an embodiment, the amount of R(+)-N-propargyl-1-aminoindan or a pharmaceutically acceptable salt thereof is effective to inhibit retinal ganglion cell death or retinal ganglion cell damage.

In an embodiment, the invention is a method of treating a subject suffering from retinal ganglion cell death or retinal ganglion cell damage an amount of R(+)-N-propargyl-1-aminoindan or a pharmaceutically acceptable salt thereof effective to decrease retinal ganglion cell death or retinal ganglion cell damage.

The invention also provides a pharmaceutical composition for use in treating a subject afflicted with glaucoma, which comprises a therapeutically effective amount of R(+)-N-propargyl-1-aminoindan or a pharmaceutically acceptable salt thereof and a pharmaceutically acceptable carrier.

The invention also provides the use of R(+)-N-propargyl-1-aminoindan or a pharmaceutically acceptable salt thereof in the manufacture of a medicament for treating a subject afflicted with glaucoma.

The invention also provides a method for reducing retinal ganglion cell death in a subject in need thereof comprising administering to the subject an amount of R(+)-N-propargyl-1-aminoindan or a pharmaceutically acceptable salt thereof effective to treat the subject.

In an embodiment, the subject suffers from increased intraocular pressure.

The present invention provides pharmaceutical compositions comprising the compound R(+)PAI, their preparations and methods of treatment of glaucoma with the pharmaceutical compositions.

Rasagiline is the INN (International Nonproprietary Name) and USAN (United States Adopted Name) of the chemical substance R(+)—N-propargyl-1-aminoindan [“R(+)PAI”].

R(+)PAI may be obtained by optical resolution of racemic mixtures of R and S-enantiomer of N-propargyl-1-aminoindan (PAI). Such a resolution can be accomplished by any conventional resolution method, well known to a person skilled in the art, such as those described in “Enantiomers, Racemates and Resolutions” by J. Jacques, A. Collet and S. Wilen, Pub. John Wiley & Sons, N.Y., 1981. For example, the resolution may be carried out by preparative chromatography on a chiral column. Another example of a suitable resolution method is the formation of diastereomeric salts with a chiral acid such as tartaric, malic, mandelic acid or N-acetyl derivatives of amino acids, such as N-acetyl leucine, followed by recrystallisation to isolate the diastereomeric salt of the desired R enantiomer.

The racemic mixture of R and S enantiomers of PAI may be prepared, e.g. as described in WO95/11016. The racemic mixture of PAI can also be prepared by reacting 1-chloroindan or 1-bromoindan with propargylamine. Alternatively, this racemate may be prepared by reacting propargylamine with 1-indanone to form the corresponding imine, followed by reduction of the carbon-nitrogen double bond of the imine with a suitable agent, such as sodium borohydride.

In accordance with this invention, R(+)PAI can also be prepared directly from the optically active R-enantiomer of 1-aminoindan by reaction with propargyl bromide or propargyl chloride in the presence of an organic or inorganic base and optionally in the presence of a suitable solvent. A preferred method of preparation of the aforementioned compound is the reaction between R-1-aminoindan with propargyl chloride using potassium bicarbonate as a base and acetonitrile as solvent.

The compound R(+)PAI may be prepared as pharmaceutical compositions particularly useful for the treatment of glaucoma. Such compositions may comprise the compound of R(+)PAI or pharmaceutically acceptable acid addition salts thereof, together with pharmaceutically acceptable carriers and/or excipients. In the practice of this invention, pharmaceutically acceptable salts include, but are not limited to, the mesylate, maleate, fumarate, tartrate, hydrobromide, esylate, p-toluenesulfonate, benzoate, acetate, phosphate and sulfate salts.

The compound R(+)PAI may be formulated into pharmaceutical compositions with pharmaceutically acceptable carriers, such as water or saline and may be formulated into eye drops, for intraocular administration.

The compositions may be prepared as medicaments to be administered orally, parenterally, rectally or transdermally. Suitable forms for oral administration include tablets, compressed or coated pills, dragees, sachets, hard or soft gelatin capsules, sublingual tablets, syrups and suspensions; for parenteral administration the invention provides ampoules or vials that include an aqueous or non-aqueous solution or emulsion; for rectal administration there are provided suppositories with hydrophilic or hydrophobic vehicles; and for topical application as ointments and transdermal delivery there are provided suitable delivery systems as known in the art.

Specific examples of pharmaceutical acceptable carriers and excipients that may be used to formulate oral dosage forms of the present invention are described, e.g., in U.S. Pat. No. 6,126,968 to Peskin et al., issued Oct. 3, 2000. Techniques and compositions for making dosage forms useful in the present invention are described in the following references: 7 Modern Pharmaceutics, Chapters 9 and 10 (Banker & Rhodes, Editors, 1979); Pharmaceutical Dosage Forms Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

The preferred dosages of R(+)PAI in any of the disclosed compositions may be within the following ranges: for oral or suppository formulations 0.01-20 mg per dosage unit to be taken daily, preferably 0.5-5 mg per dosage unit to be taken daily and more preferably 1 mg or 2 mg per dosage unit to be taken daily may be used.

For topical ocular administration the novel formulations of this invention may take the form of solutions, gels, ointments, suspensions or solid inserts, formulated so that a unit dosage comprises a therapeutically effective amount of the active component or of a combination therapy. For ocular administration, 0.01-2 mg per dosage unit to be taken daily, preferably 0.1-1 mg per dosage unit, or a pharmaceutically acceptable salt, to be taken daily may be used.

The pharmaceutical preparation may contain the any of the following non-toxic auxiliary substances:

The pharmaceutical preparation may contain antibacterial components which are non-injurious in use, for example, thimerosal, benzalkonium chloride, methyl and propyl paraben, benzyldodecinium bromide, benzyl alcohol, or phenylethanol.

The pharmaceutical preparation may also contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The pharmaceutical preparation may also contain a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers.

The pharmaceutical preparation may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like.

The pharmaceutical preparation may also contain surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold, for example, by BASF under the trademark Cremaphor.

The pharmaceutical preparation may also contain wetting agents commonly used in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears, wherein the wetting agent is present in an amount of about 0.001% to about 10%.

The formulation of this invention may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium edetate; and drug solubility enhancers, such as polyethylene glycols. These additional ingredients help make commercial solutions with adequate stability so that they need not be compounded on demand.

Ophthalmic compositions will be formulated so as to be compatible with the eye and/or contact lenses to be treated with the compositions. As will be appreciated by those skilled in the art, the ophthalmic compositions intended for direct application to the eye will be formulated so as to have a pH and tonicity which are compatible with the eye. This will normally require a buffer to maintain the pH of the composition at or near physiologic pH (i.e., 7.4) and may require a tonicity agent to bring the osmolality of the composition to a level at or near 210-320 milliosmoles per kilogram (mOsm/kg).

The dose can be appropriately selected depending upon symptom, age, dosage form, etc. and, in the ophthalmic solutions, contain between 0.02-2 mg per day of R(+)PAI, preferably between 0.1 to 1 mg per day of R(+)PAI or a pharmaceutically acceptable salt in a pharmaceutically acceptable ophthalmic carrier. The pH can be within a range which is acceptable to ophthalmic preparations and, preferably within a range from 4 to 8.

Other materials as well as processing techniques and the like are set forth in Part 8 of Remington's Pharmaceutical Sciences, 17th edition, 1985, Mack Publishing Company, Easton, Pa., and International Programme on Chemical Safety (IPCS), which is incorporated herein by reference.

There are a number of non-invasive or minimally invasive drug delivery techniques that are suitable for delivery of a drug into the posterior segment of a subject's eye. Four approaches may be used to deliver drugs to the posterior segment—topical, systemic, intraocular, and periocular (including subconjunctival, sub-Tenon's, and retrobulbar). It should be noted that any means of administering compounds to the eye of a subject should be considered to be within the scope of the present invention. In one aspect, for example, solutions and suspensions that can be administered in the form of drops can be used. In other aspect, agents may also be administered via intravitreal, periocular or subconjunctival injection, application of ultrasound to the eye, by microporation with microneedles, or scleral implantation. In yet another aspect, iontophoretic devices and methods may be used to non-invasively administer drugs into the eye that may be particularly successful in achieving a high degree of drug penetration with a short duration. Therefore, subject discomfort and inconvenience are minimized, as well as the risk of certain potential adverse side effects for the treatment regimen as a whole.

R(+)PAI compositions may be used alone to treat glaucoma, or alternatively, they may be used as an adjunct to existing glaucoma treatments.

By any range disclosed herein, it is meant that all hundredth, tenth and integer unit amounts within the range are specifically disclosed as part of the invention. Thus, for example, 0.01-20 mg means that 0.01, 0.02, 0.03 . . . 0.09; 0.1, 0.2 . . . 0.9; and 1, 2 . . . 19 and 20 mg unit amounts are included as embodiments of this invention.

As used herein, a subject “afflicted” with glaucoma means the subject has been diagnosed with glaucoma.

### Experimental Details

Degeneration of retinal ganglion cells (RGCS), axon loss and an excavated appearance of the optic nerve head are associated with glaucoma. In recent years there has been increasing interest in preventing progression of glaucomatous optic neuropathy using approaches based on the premise that glaucoma is a neurodegenerative disease (Fisher, et al., “Vaccination for Neuroprotection in the Mouse Optic Nerve: Implications for Optic Neuropathies”, J Neurosci 2001; 21:136-142). Neuroprotection of the glaucomatous optic nerve could therefore be an adjunctive therapeutic paradigm for use with conventional IOP-lowering treatments (Schwartz, et al., “Potential Treatment Modalities for Glaucomatous Neuropathy: Neuroprotection and Neurodegeneration”, J. Glaucoma 1996; 5:427-432). Neuroprotection is a novel therapeutic paradigm for slowing or preventing degeneration and death of neurons to maintain their physiological function. An important advantage of the neuroprotective strategy is that it allows treatment of disease for which the specific etiology is either unknown or differs among patients. This is particularly relevant to the treatment of glaucoma where neuroprotection should be effective independently of whether a particular patient's glaucoma is due to primary or secondary disease mechanisms (weinreb, et al., “Is Neuroprotection a Viable Therapy for Glaucoma?”, Arch Opthalmol 1999; 117:1540-1544). Though significantly decreasing neuronal loss, the current IOP-lowering medications do not halt the progressive nature of glaucoma, and the loss of RGCs may continue even after the IOP has been reduced. Thus, the greatest unmet medical need in glaucoma is a therapeutic agent capable of protecting ocular tissue from continued degeneration.

The effect of rasagiline on RGC survival is tested in a rat model of chronically elevated IOP, a major risk factor in glaucoma.

### EXAMPLE 1

### Ocular Hypertension as Model for Glaucoma

Glaucoma is commonly linked to raised intraocular pressure (IOP), the precise means by which IOP may lead to RGC apoptosis. In this well-established glaucoma model an elevated IOP is caused by surgically induced chronic ocular hypertension (OHT). (Guo et al. “Targeting amyloid-β in glaucoma treatment”, PNAS 2007; 104:113444-13449)

### Rasagiline

Rasagiline was obtained as its mesylate salt (1 mg salt is equivalent to 0.64 mg free base).

### Materials and Methods

A unilateral increase in IOP is induced in anesthetized male Lewis rats by laser photocoagulation of the limbal and episcleral veins. Rats receive two laser cauterization treatments, one week apart. IOP is measured one week following the second laser treatment. The second laser treatment is followed two weeks later by application of a fluorescent retrograde neurotracer distally to the optic nerve head. One day after dye application (3 weeks after the initial laser treatment) the rats are sacrificed, their retinas excised, fixed in paraformaldehyde and whole mounted on filters. Survival of RGCs is determined by counting the labeled cells using a fluorescent microscope.

To examine the effect of rasagiline on the survival of RGCs, rats receive a single subcutaneous injection of rasagiline prior to the second laser treatment. A further group of naïve animals receives no laser treatments. The “% Protection” of treatment with rasagiline in relation to control (PBS) treatment, and the statistical significance of the effect, are calculated.

### Repeated Injections—Treatment Protocol

To examine the effect of rasagiline weekly and monthly treatment on the survival of RGCs, rats receive repeated subcutaneous injection of rasagiline for 12 weeks, starting on the day of the second laser treatment. A control group receives weekly PBS, additional positive control (PC) group receives a single injection of rasagiline on the day of the second laser treatment (Day +7).

### Repeated Injections—Prevention Protocol

To examine the effect of rasagiline weekly and monthly treatment on the survival of RGCs, rats receive repeated subcutaneous injection of rasagiline for 12 weeks, starting prior to the second laser treatment, last injection for all groups on the day of the second laser treatment. A control group receives weekly PBS, additional positive control (PC) group receives a single injection rasagiline on the day of the second laser treatment.

### Results

Rasagiline shows a positive effect in the IOP model under the protocols tested.

### EXAMPLE 2

### Rat Model of Chronic, Moderately Elevated IOP

The effect of rasagiline on RGC survival is tested in a rat model of chronic, moderately elevated IOP, a major risk factor in glaucoma. Because elevated IOP is a major risk factor for progression of glaucoma, treatment has been based on lowering IOP. The following is rat model of chronic, moderately elevated IOP. (Nuefeld et al. “Inhibition of nitric-oxide synthase 2 by aminoguanidine provides neuroprotection of retinal ganglion cells in a rat model of chronic glaucoma” PNAS, 1999, 96: 9944-9948)

### Materials and Methods

Chronic, moderately elevated IOP is produced unilaterally by cautery of three episcleral vessels; the contralateral eye served as the control. To perform the cautery, sutures are placed in the lids to keep the eye open and in the bulbar conjunctiva to manipulate the globe. Three of the four to five major trunks formed by limbal-derived veins are exposed at the equator of the eye by incising the conjunctiva. Each vessel is lifted with a small muscle hook and cauterized by direct application of an ophthalmic, disposable cautery against the muscle hook. Immediate retraction and absence of bleeding of the cauterized ends of the vessels are noted as successful cauterization. After surgery, eyes are treated topically with bacitracin-neomycin-polymyxin for a few days during recovery.

One group is treated with rasagiline, in the drinking water, for a period of time. Another group is not treated with rasagiline, but receives drinking water on the same schedule. Once a month, each animal is anesthetized and IOP is determined bilaterally. The animals are awake within 15 minutes of the IOP measurement. On any given eye, three to five tonometer readings are taken and averaged. After six months of unilateral, chronic, moderately elevated IOP, photographs are taken of the optic disks of each eye of the anesthetized rats with a fundus camera through a coverslip placed on the cornea with a drop of Gonisol.

One week before sacrifice, Fluoro-Gold is microinjected bilaterally into the superior colliculi of anesthetized rats immobilized in a stereotaxic apparatus. Fluoro-Gold is taken up by the axon terminals of the retinal ganglion cells and bilaterally transported retrogradely to the somas in the retina. One week after Fluoro-Gold application, animals are sacrificed by overdose of the above anesthetic mixture and whole, flat-mounted retinas are assayed for retinal ganglion cell density. Rat eyes are enucleated and fixed in 4% paraformaldehyde for 30 minutes. Eyes are bisected at the equator, the lens is removed, and the posterior segments are prepared for flat mounts. Retinas are dissected from the underlying sclera, flatted by six radial cuts, and mounted vitreal side up on gelatin-coated slides.

Labeled retinal ganglion cells are counted using fluorescence microscopy in 12 fields of the retina.

### Results

Rasagiline shows a positive effect in the chronic, moderately elevated IOP model under the protocols tested.

### EXAMPLE 3

### Staurosporine (SSP) Model for Glaucoma

The effect of rasagiline on RGC survival is tested in a rat model of RGC survival.

Staurosporine (Streptomyces staurospores) is a relatively non-selective protein kinase inhibitor, which blocks many kinases to different degrees. Staurosporine is often used as a general method for inducing apoptosis. In this well-established model it is used to induce apoptosis of retinal ganglion cells. (Cordeiro, et al. “Real-time imaging single nerve cell apoptosis in retinal neurodegeneration” PNAS, 2004, 202:13352-13356)

### Animals

Adult rats are used in all rat experiments.

### Materials and Methods

Rats receive a dose of intravitreal SSP in PBS. Animals are imaged immediately and up to 6 h, after which they are sacrificed for histology.

To examine the effect of rasagiline on the survival of RGCs, one group of rats are treated with rasagiline for a period of time before SSP. A further group of naïve rats receives no rasagiline.

Imaging with Alexa Fluor 488-Labeled Annexin 5

The animal is positioned before the cLSO so that the interior of the eye is imaged. An Argon laser wavelength of 488 nm is focused into a small spot and scanned across the retina by a pair of mirrors to excite the administered annexin 5-bound fluorophore. The fluorescence is detected by a solid-state photodetector.

For imaging, animals are held in a stereotaxic frame and their pupils dilated. Videos of scanned retinal areas are assessed for fluorescence. All animals have baseline images recorded before receiving intravitreal injections of Alexa Fluor 488-labeled annexin-5.

### Histology

After killing, eyes are enucleated and fixed immediately in 4% fresh paraformaldehyde, after which they are dissected at the equator, the lens and vitreous are removed, and whole flat retinas are obtained.

### Apoptosis Identification

Whole retinas are blocked for 2 h and incubated with selected antibodies. After washing in PBS, the retinas are flattened by four radial cuts and mounted vitreal side up with glycerol/PBS solution. Flat retinas are also processed for frozen sections.

### RGC Identification

To identify RGCs, whole flat retinas and frozen sections are stained to assess nuclei.

### Confocal Microscopy

Fluorescent retinas are assessed by using a confocal laser scanning microscope.

### Image Analysis

The number of stained RGC and annexin 5-labeled apoptotic RGCs are counted with microscopy analysis software.

### Results

Rasagiline shows a positive effect in the SSP model under the protocols tested.

### EXAMPLE 4

### The Effect of Rasagiline on the Survival of Retinal Ganglion Cells in Rats with Experimental Glaucoma

The purpose of this example is to find out if rasagiline is neuroprotective in an established model of experimental glaucoma in rats.

### Materials and Methods

Male Wistar rats weighing 375-400 gm are treated under procedures approved and monitored by the Animal Care Committee of the Tel-Aviv University School of Medicine and following the procedures outlined in the Association for Research in Vision and Opthalmology Statement for the use of animals in ophthalmic and vision research. Animals are housed with a 14 hour light/10 hour dark cycle with standard chow and water ad libitum.

Glaucoma is induced in one eye of rats by using the translimbal laser photocoagulation model developed by Levkovitch-Verbin. This model can produce elevated IOP and typical glaucomatous optic nerve damage in most treated eyes.

In this model, the outflow channels of the rat eye are treated by argon laser at 532 nm. Animals are anesthetized with intraperitoneal ketamine (10-13 mg/kg) and xylazine (50 mg/kg) and topical proparacaine 1% eye drops. The laser treatment is given unilaterally to the left eye and this is repeated after one week. IOP is measured with Tonopen XL under the above anaesthesia in both eyes before and immediately after laser treatment and weekly thereafter. Each time ten measurements are obtained on each eye and the mean value is calculated. The retinal and choroidal blood vessels are observed by indirect opthalmoscopy to assure the patency of vessels, and to identify retinal edema or hemorrhage.

Rasagiline is administered intraperitoneally, starting immediately after the laser treatment at two dose levels: 0.5 and 3 mg/kg. The compound is applied once daily, until the end of the experiment, for the duration of 6 weeks. The volume administration is 2 ml/kg (in saline). The drug injections are performed for 5 working days. Each group includes 15 rats:

1. Laser treated and vehicle saline (IP)
2. Laser treated and TCG 0.5 mg/kg (IP)
3. Laser treated and TCG 3 mg/kg (IP)

Rasagiline is administered IP daily for 6 weeks. (5 working days) Rasagiline is prepared by dissolving saline at 0.5 mg/kg and 3.0 mg/kg. The volume of administration is 2 ml/kg. Rasagiline can be prepared once at the beginning of each week, for 0.5 mg/kg at 20 mg/80 ml and for 3.0 mg/kg at 120 mg/80 ml. (calculated for a rat weighing 400 g). The solution is kept in the refrigerator at −4° C. Each week a fresh solution is prepared.

Ten days before sacrifice retinal ganglion cells are labelled by applying fluorescent dye (Fluorogold) to the superior colliculus by stereotactic injections, bilaterally. Upon sacrifice all animal are anesthetized and eyes are removed. Retinal whole mounts are placed on slides.

Thirty-two (32) images of ×40 magnification from each retina (both eyes for each rat) are photographed using a fluorescent microscope, and the number of surviving retinal ganglion cells are counted for each eye. The number of RGCs in the experimental eye are compared to the fellow control eye to calculated the RGC loss. The RGCs are counted by a blinded observer.

### Results

Rasagiline shows a positive effect in the experimental glaucoma model under the protocols tested. More ganglion survival is evident in the treated group. This indicates that treatment with rasagiline eliminates processes which contribute to ganglion death.

### EXAMPLE 5

### Screening for Efficacy in Reducing the Photoreceptors Damage using a Rat Retinal Ischemia/Reperfusion Model—Intraperitoneal Administration/Ocular Hyperpressure

The objective of this study is to determine whether intraperitoneal (IP) administration of rasagiline results in a better recovery of the retinal electric activity and/or a decrease of apoptotic retinal ganglion cells (RGCs) after a transient ischemia induced by ocular hyperpressure.

### Materials and Methods

Thirty (30) male pigmented rats (Long Evans) are obtained and divided evenly into 3 groups, (2 test groups and 1 control group) 10 animals in each group. Retinal ischemia is induced by a 100/200-mmHg hyperpressure saline column applied to the eye through a needle for 60 minutes.

For the two test groups, rasagiline (0.5 and 3 mg/mg solution in vehicle) is administered IP 30 minutes before ischemia and 2 hours after ischemia, then once daily until termination of the study. For the control group, the vehicle (saline solution) is administered IP 30 minutes before ischemia and 2 hours after ischemia.

### Measurements

Electroretinographic (ERG) measurements of a- and b-wave implicit times and peak amplitudes under scotopic conditions at maximal intensity are taken at baseline (just before ischemia), and 4-7 days after reperfusion.

### Histology

Sampling of fixed flatmount retinas and histological processing for annexin-5 labeling or TUNEL labeling.

### Results

Intraperitoneal (IP) administration of rasagiline has a positive effect on the recovery of the retinal electric activity and/or decreases the number of apoptotic RGCs after transient ischemia induced by ocular hyperpressure.

### EXAMPLE 6

### MAO Activity and Inhibition in Rat Brains, Livers and Retinas after Rasagiline Intraocular or P.O Administration for 10 Days

The aim of this study were 1) to present evidence that rasagiline, administered in the form of eye drops, penetrates the inner layers of the eye by examining the extent of MAO inhibition in the retina, and 2) to determine rasagiline doses which inhibit MAO-A and MAO-B in the retina and assess the systemic penetration of rasagiline by examining the extent of MAO inhibition in internal organs like liver and brain.

### Materials and Methods

Male SPF Sprague Dawely Rats within the weight range of 270±7 grams were used in this study.

Rasagiline eye formulation was prepared every 2 days in the following concentrations: 60, 20, 4, 0.8, 0.16 mg/ml (base). The formulation of 60 mg/ml solution was prepared in water for injection only and the 20-0.16 mg/ml concentrations were prepared in 50 mg/ml mannitol solution in order to keep appropriate osmolarity (280-610 mOsmol/kg). pH range was 4.38-5.59.

About 15-18 μl from those preparations were applied to each eye of rats, 6 in a group, according to the group average weight.

The solution of 50 mg/kg mannitol was used as the vehicle control group for eyes treatment. The rats that received the intraocular treatment were anesthetized with isoflurane before administration to the eyes.

Rasagiline for P.O. administration was prepared in DDW. Water was administered to the P.O vehicle control group.

Rasagiline was administered for 10 days. Rats were sacrificed 2-3 hours after last administration. According to preliminary experiment, in order to obtain enough substance for MAO and protein analysis, 4 retinas (from 2 rats) were combined into one sample, (3 retina samples for MAO per each dose groups). The 6 brains and 6 livers were analyzed separately. In a preliminary test performed for 2 days it was found that rasagiline did not cause irritation to the eye.

The dose groups are shown in the following table:

TABLE 1
Study Design

| | | | | Conc. | Volume | Obtained |
|---|---|---|---|---|---|---|
| Group | Rats | | | mg/ml | Per eye | dose |
| # | # | group | route | base | μl | mg/kg/day |
| 1 | 1-6 | Control | Eye drops | | 14-15 | — |
| | | 50 mg/ml | | | | |
| | | mannitol | | | | |
| 2 | 7-12 | rasagiline | Eye drops | 60 | 18-18.5 | 7.9 |
| 3 | 13-18 | rasagiline | Eye drops | 20 | 14-15 | 2.0 |
| 4 | 19-24 | rasagiline | Eye drops | 4 | 14-15 | 0.4 |
| 5 | 25-30 | rasagiline | Eye drops | 0.8 | 14-15 | 0.08 |
| 6 | 31-36 | rasagiline | Eye drops | 0.16 | 14-15 | 0.165 |
| 7 | 37-42 | rasagiline | P.O | 0.1 | 1.1-1.2 ml | 0.41 |
| 8 | 43-48 | Control | P.O | | 1.1-1.2 ml | — |
| | | water | | | | |

The standard method was used for the enzymatic determination of MAO, IRD-MB-051: “Determination of monoamine oxidase (MAO) by an extraction method using radiolabelled substrate in various tissues”.

### Results

TABLE 2
MAO Activity in Rats Brains, Livers and Retinas after Rasagiline
Intraocular or P.O Administration for Ten Days

| | Dose | Brain | | Liver | | Retina | |
|---|---|---|---|---|---|---|---|
| Group | Mg/kg/day | MAO-A | MAO-B | MAO-A | MAO-B | MAO-A | MAO-B |
| 1 | Mannitol | 8375 ± 331 | 2398 ± 338 | 5940 ± 534 | 1537 ± 339 | 1312 ± 534 | 652 ± 235 |
| 2 | 7.9 | 242 ± 101 | 18 ± 6 | 378 ± 54 | 32 ± 8 | 38 ± 10 | 12 ± 8 |
| 3 | 2.0 | 580 ± 101 | 24 ± 9 | 1381 ± 130 | 71 ± 9 | 56 ± 19 | 15 ± 2 |
| 4 | 0.4 | 2958 ± 509 | 52 ± 6 | 3527 ± 335 | 113 ± 14 | 435 ± 126 | 11 ± 9 |
| 5 | 0.08 | 6524 ± 180 | 125 ± 28 | 5499 ± 838 | 415 ± 63 | 849 ± 136 | 25 ± 3 |
| 6 | 0.0165 | 7631 ± 342 | 713 ± 95 | 6237 ± 337 | 1414 ± 286 | 1690 ± 215 | 60 ± 24 |
| 8 | Water | 8360 ± 247 | 8633 ± 839 | 5892 ± 442 | 6321 ± 645 | 1614 ± 181 | 1202 ± 285 |
| (P.O) | | | | | | | |
| 7 | 0.41 | 4437 ± 397 | 164 ± 15 | 2855 ± 384 | 113 ± 19 | 968 ± 147 | 28 ± 10 |

TABLE 3
Percent of MAO-A and MAO-B Inhibition in Rat Brains, Livers and Retinas
after Rasagiline Intraocular or P.O. Administration for Ten Days

| | Dose | Brain | | Liver | | Retina | |
|---|---|---|---|---|---|---|---|
| Group | Mg/kg/day | MAO-A | MAO-B | MAO-A | MAO-B | MAO-A | MAO-B |
| 1 | Mannitol | 0 | 0 | 0 | 0 | 0 | 0 |
| 2 | 7.9 | 97 | 99 | 94 | 98 | 97 | 98 |
| 3 | 2.0 | 93 | 99 | 77 | 95 | 96 | 98 |
| 4 | 0.4 | 65 | 98 | 41 | 93 | 67 | 98 |
| 5 | 0.08 | 22 | 95 | 7 | 73 | 35 | 96 |
| 6 | 0.0165 | 9 | 70 | −5 | 8 | −29 | 91 |
| 8 | Water | 0 | 0 | 0 | 0 | 0 | 0 |
| (P.O) | | | | | | | |
| 7 | 0.41 | 47 | 98 | 52 | 98 | 40 | 98 |
| (P.O) | | | | | | | |

TABLE 4
MAO-A Activity and Percent Inhibition in Rat's Retina after Ten Days -
Rasagiilne Intraocular or P.O Administration - Comparison of Percent
Inhibition Calculated from dpm and from Activity (nmol/hour/mg protein)

| | | MAO-A | | | |
|---|---|---|---|---|---|
| | Dose | | % | MAO-A | % |
| | Mg/kg/ | MAO-A | Inhi- | nmol/hour/mg | Inhi- |
| Group | day | dpm ± sd | bition | protein ± sd | bition |
| 1 | Manni- | 1312 ± 534 | 0 | 12.01 ± 3.43 | 0 |
| | tol | | | | |
| 2 | 7.9 | 38 ± 10 | 97 | 0.4 ± 0.03 | 97 |
| 3 | 2.0 | 56 ± 19 | 96 | 0.59 ± 0.1 | 95 |
| 4 | 0.4 | 435 ± 126 | 67 | 3.88 ± 0.59 | 68 |
| 5 | 0.08 | 849 ± 136 | 35 | 10.33 ± 0.89 | 14 |
| 6 | 0.0165 | 1690 ± 215 | −29 | 19.89 ± 0.4 | −66 |
| 8 | Water | 1614 ± 181 | 0 | 23.93 ± 2.17 | 0 |
| (P.O) | | | | | |
| 7 | 0.41 | 968 ± 147 | 40 | 11.06 ± .0.21 | 54 |
| (P.O) | | | | | |

Group result is an average of 3 samples. Each sample contains 4 retinas (from 2 rats); Percent Inhibition were calculated in comparison to the controls

TABLE 5
MAO-B Activity and Percent Inhibition in Rat's Retina after Ten Days -
Rasagiilne Intraocular or P.O Administration - Comparison of Percent
Inhibition Calculated from dpm and from Activity (nmol/hour/mg protein)

| | | MAO-B | | | |
|---|---|---|---|---|---|
| | Dose | | % | MAO-B | % |
| | Mg/kg/ | MAO-B | Inhi- | nmol/hour/mg | Inhi- |
| Group | day | dpm ± sd | bition | protein ± sd | bition |
| 1 | Manni- | 625 ± 235 | 0 | 0.8 ± 0.18 | 0 |
| | tol | | | | |
| 2 | 7.9 | 12 ± 8 | 98 | 0.02 ± 0.01 | 98 |
| 3 | 2.0 | 15 ± 2 | 98 | 0.02 ± 0.00 | 97 |
| 4 | 0.4 | 11 ± 9 | 98 | 0.01 ± 0.01 | 98 |
| 5 | 0.08 | 25 ± 3 | 96 | 0.04 ± 0.01 | 95 |
| 6 | 0.0165 | 60 ± 24 | 91 | 0.10 ± 0.04 | 88 |
| 8 | Water | 1202 ± 285 | 0 | 2.41 ± 0.67 | 0 |
| (P.O) | | | | | |
| 7 | 0.41 | 28 ± 10 | 98 | 0.04 ± 0.01 | 98 |
| (P.O) | | | | | |

Group result is an average of 3 samples. Each sample contains 4 retinas (from 2 rats); Percent Inhibition were calculated in comparison to the controls

### Discussion

Rasagiline administered intraocularly was absorbed in all doses. A vehicle effect of mannitol on MAO-B activity was observed in all tissues tested. Table 2 shows that MAO-B activity in the brain, liver and retina of rats treated intraocularly with mannitol (isofluran anesthesia) is 72%, 76% and 46% respectively lower than MAO-B activity in brains of rats treated orally with water. In addition, Table 5 shows that MAO-B activity in retinas of rats treated intraocularly with mannitol is 46% or 67% lower than MAO-B activity in retinas of rats treated orally with water, calculated from dpm and from activity per protein respectively.

Since the weight of a retina is very low, protein content was determined in order to decide whether results after normalization to protein would be more accurate. It was observed that similar percent inhibition was obtained when calculated from dpm results and from activity. Results of percent inhibition in retina calculated from activity and from dpm are similar, suggesting that protein analysis can be omitted in future experiments allowing the use of 2 retinas per sample instead of 4 retinas per sample.

Finally, it was observed that systemic inhibition depends on dose and that dose dependency varies from tissue to tissue. For example, a dosage of 0.0165 mg/kg/day does not inhibit liver MAO-B but does inhibit brain MAO-B, possibly via optic nerve. (See Table 3) In addition, 0.4 mg/kg/day of intraocular rasagiline causes slightly higher MAO-A inhibition in retina and brain in comparison to 0.4 mg/kg/day given orally. (See Table 3) The results show that local MAO inhibition in the retinas was higher than the level of inhibition observed in the liver and inhibition of the brain enzymes was similar to that of the retina enzymes.

This example shows that the risk of MAO-A reaching the liver from intraocular administration is less than that of oral administration. This result suggests that the risk of the “cheese effect” commonly associated with MAO inhibitors is less in the case of intraocular administration as compared to the same in the case of oral administration.

### EXAMPLE 7

### Clinical Testing

Based on the foregoing, a clinical trial is undertaken.

### Glaucoma Clinical Trial

A multi-center, randomized double-blind, placebo-controlled, multiple-dose, three-arm study to assess the tolerability, safety and the efficacy of rasagiline in patients with glaucomatous optic neuropathy. Each subject receives placebo or rasagiline.

### Results

Patients treated with rasagiline demonstrate increased protection against loss of RGCs and consequent reduced severity of glaucoma symptoms, e.g. reduced atrophy of the optic nerve, as compared to the group receiving the placebo. The patients receiving rasagiline also demonstrate reduced visual field loss and increased preservation of the retina and of the structural integrity of the optic nerve.

1. A method of treating a subject afflicted with glaucoma, comprising administering to the subject an amount of R(+)-N-propargyl-1-aminoindan or a pharmaceutically acceptable salt thereof effective to treat the subject.

2. A method of treating a subject suffering from retinal ganglion cell death or retinal ganglion cell damage, or of reducing retinal ganglion cell death or damage in a subject, comprising administering to the subject an amount of R(+)-N-propargyl-1-aminoindan or a pharmaceutically acceptable salt thereof effective to reduce retinal ganglion cell death or retinal ganglion cell damage.

3. The method of claim 2, wherein the subject suffers from increased intraocular pressure.

4. The method of claim 1, wherein the amount of R(+)-N-propargyl-1-aminoindan or of the pharmaceutically acceptable salt thereof is from 0.01 mg to 20 mg per day.

5. The method of claim 4, wherein the amount of R(+)-N-propargyl-1-aminoindan or of the pharmaceutically acceptable salt thereof is from 0.5 mg to 5 mg per day.

6. The method of claim 4, wherein the amount of R(+)-N-propargyl-1-aminoindan or of the pharmaceutically acceptable salt thereof is 2 mg per day.

7. The method of claim 4, wherein the amount of R(+)-N-propargyl-1-aminoindan or of the pharmaceutically acceptable salt thereof is 1 mg per day.

8. The method of claim 1, wherein the administration is of the pharmaceutically acceptable salt of R(+)-N-propargyl-1-aminoindan.

9. The method of claim 8, wherein the pharmaceutically acceptable salt is esylate, mesylate, sulfate or tartrate.

10. The method of claim 9, wherein the pharmaceutically acceptable salt is mesylate.

11. The method of claim 10, wherein the amount of R(+)-N-propargyl-1-aminoindan mesylate is 1.56 mg per day.

12. The method of claim 1, wherein the administration is intraocular, ocular, oral, parenteral, periocular, rectal, systemic, topical or transdermal administration.

13. The method of claim 12, wherein the administration is ocular.

14. The method of claim 12, wherein the administration is to the posterior segment.

15. The method of claim 14, wherein the administration is intraocular, periocular, systemic or topical.

16. The method of claim 13, wherein the amount of R(+)-N-propargyl-1-aminoindan mesylate is from 0.01 mg to 2 mg per day.

17. The method of claim 13, wherein the amount of R(+)-N-propargyl-1-aminoindan mesylate is from 0.1 mg to 1 mg per day.

18. The method of claim 1, wherein the R(+)-N-propargyl-1-aminoindan or the pharmaceutically acceptable salt thereof is in a pharmaceutical composition.

19. The method of claim 1, further comprising administering to the subject an additional agent for treating glaucoma.

20. The method of claim 19, wherein the additional agent for treating glaucoma is a β-adrenergic antagonist, adrenergic agonist, parasympathomimetic, prostaglandin-like analog, or carbonic anhydrase inhibitor.

21. The method of claim, wherein the amount of R(+)-N-propargyl-1-aminoindan or a pharmaceutically acceptable salt thereof is effective to inhibit retinal ganglion cell death or retinal ganglion cell damage.

22. A pharmaceutical composition comprising R(+)-N-propargyl-1-aminoindan or a pharmaceutically acceptable salt thereof, an additional agent for treating glaucoma, and a pharmaceutically acceptable carrier.

23. The pharmaceutical composition of claim 22, wherein the agent for treating glaucoma is a β-adrenergic antagonist, adrenergic agonist, parasympathomimetic, prostaglandin-like analog, or carbonic anhydrase inhibitor.

24-26. (canceled)
